(19) United States
(12) Patent Application Publication
(10) Pub. No.: US 2012/0058961 A1
(43) Pub. Date: Mar. 8, 2012

(54) Pharmaceutical Composition for Treating Cancers

(75) Inventor: John Soong, Chino, CA (US)

(73) Assignee: HENKAN PHARMACEUTICAL CO., LTD., Taipei City (TW)

(21) Appl. No.: 12/877,380

(22) Filed: Sep. 8, 2010

(51) Int. Cl. A61K 31/7048; A61P 35/00

(52) U.S. Cl. 514/26

(57) ABSTRACT

The preset invention relates to a new approach for treating cancer by using a synergistic combination of a histone deacetylase inhibitor and a furost-5-ene-3,22,26-triol glycoside. A pharmaceutical composition for treating a cancer comprising the synergistic combination and the treatment of a cancer with the synergistic combination are provided.

### FIELD OF THE INVENTION

The present invention relates to a new pharmaceutical composition for treating cancers with enhanced anti-cancer efficacy.

### BACKGROUND OF THE INVENTION

Histone deacetylase inhibitors (HDACi) can induce diverse biological responses in tumor cells including induction of apoptosis, and suppression of cell proliferation, and combining HDACi with other pro-apoptotic agents can result in synergistic apoptosis and superior anti-tumor activities compound to those observed using single agents (Bolden et al., Anticancer activities of histone deacetulase inhibitors, Nat. Rev. Drug Discov. 5: 769-784, 2006).

Vorinostat is the first HDACi to be approved for clinical use in treating patients with malignancy (e.g., cutaneous T-cell lymphoma). A hypothesis was provided by Dokmanovic et al. that the rapid reversal of the binding of the HDACi to its target may provide normal cells with the ability to compensate for the inhibitory effects of these agents, whereas cancer cells with multiple defects altering proteins regulating cell proliferation, survival, death, and migration are less able to compensate for the effect of the HDACi (Dokmanovic et al., Histone deacetylase inhibitors: overview and perspectives, Mol. Cancer Res. 5(10): 981-989, 2007). If the hypothesis is valid, clinical therapeutic strategies involving intermittent dosing may be a most effective regimen to achieve selective anticancer activity, and a therapeutic strategy using HDACi with other anticancer agents may be most promising.

It is also suggested by Frew et al. that HDACi are promising anti-cancer agents and provide synergistic tumor cell killing efficacy in combination with some anticancer drugs, but HDACi may act to augment the inhibitory effect of compounds that target specific kinases in some instances (Frew et al., Enhancing the apoptotic and therapeutic effects of HDAC inhibitors, Cancer Letters 280:125-133, 2009).

It is desirable to have a new approach for treating cancer with enhanced anticancer efficacy and safety.

### SUMMARY OF THE INVENTION

The present invention relates to a new approach for treating cancers by using a combination of a histone deacetylase inhibitor. It is unexpectedly discovered in the present application that a combination of a histone deacetylase inhibitor and a furost-5-ene-3,22,26-triol glycoside provides a synergistic anticancer efficacy.

In one aspect, the invention provides a pharmaceutical composition for treating cancer comprising a synergistic combination of a histone deacetylase inhibitor and a furost-5-ene-3,22,26-triol glycoside having a structure shown in formula I:

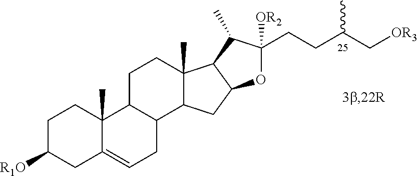

wherein R₁ is one selected from the group consisting of hydrogen, a glucose, a rhamnose, a galactose, a xylose, an arabinose, a tetra-saccharide, a penta-saccharide, and a hexa-saccharide, wherein the tetra-saccharide, penta-saccharide, and hexa-saccharide are each composed of monosaccharides selected from glucose, rhamnose, galactose, xylose and arabinose (wherein the stereo configuration at C-25 is either R (rectus) form or S (sinister) form); R₂ is hydrogen and methyl group, and R₃ is one selected from the group consisting of hydrogen, a glucose, a rhamnose, a galactose, a xylose, and an arabinose;
and which comprises a therapeutically effective amount of the synergistic combination, and a pharmaceutically acceptable carrier.

In one example of the invention, the histone deacetylase inhibitor is sodium butyrate (NaB), or suberoylanilide hydroxamic acid (SAHA).

In the other aspect, the invention provides a method for treating a cancer comprising administering to a subject in need thereof a therapeutically effective amount of a synergistic combination of a histone deacetylase inhibitor and a furost-5-ene-3,22,26-triol glycoside of formula I with a pharmaceutically acceptable carrier, simultaneously or subsequently.

### BRIEF DESCRIPTION OF THE DRAWINGS

The present invention may be better understood by reference to the following examples when considered in conjunction with the drawings. In the drawings,

FIG. 1A illustrates the results of the tests when the cells treated with 1 mM NaB, 8 uM HK-06-D01, its combination as test compounds and controls, wherein the means of Apoptosis % being 97.33% when the cells were treated with a combination of 1 mM NaB and 8 uM HK-06-D01 is much higher than that of Apoptosis % being 29.67% when the cells were treated with 1 mM NaB only (p<0.01), or that of Apoptosis % being 46.67% when the cells were treated with 8 uM HK-06-D01 only (p<0.05);

FIG. 1B illustrates the results of the tests when the cells treated with 0.3 mM NaB, 8 uM HK-06-D01, its combination as test compounds and controls, wherein the means of Apoptosis % being 72% when the cells were treated with a combination of 0.3 mM NaB and 8 uM HK-06-D01, is much higher than the means of Apoptosis % being 4% when the cells were treated with 0.3 mM NaB only, or that of Apoptosis % being 51% when the cells were treated with 8 uM HK-06-D01 only;

FIG. 1C illustrates the results of the tests when the cells treated with 0.6 mM NaB, 3 uM HK-06-D01, its combination as test compounds and controls, wherein the means of Apoptosis % being 27% when the cells were treated with a combination of 0.6 mM NaB and 3 uM HK-06-D01, is much higher than the means of Apoptosis % being 3% when the cells were treated with 0.3 mM NaB only, or that of Apoptosis % being 5% when the cells were treated with 8 uM HK-06-D01 only;

FIG. 2A illustrates the dose response in Apoptosis % for each of SAHA and the combination of SAHA and HK-06-D01; and

FIG. 2B provides an isobologram for each of SAHA and the combination of SAHA and HK-06-D01, indicating that the combination of SAHA and HK-06-D01 provided a synergistic efficacy in inducing apoptosis in cancer cells, much more than an additive efficacy.

### DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereof known to those skilled in the art.

The present invention provide a pharmaceutical composition for the treating a cancer comprising a synergistic combination of a histone deacetylase inhibitor and a furost-5-ene-3,22,26-triol glycoside. The furost-5-ene-3,22,26-triol glycoside has a structure shown in formula I:

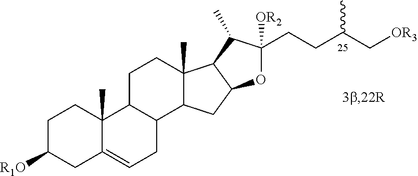

wherein R₁ is one selected from the group consisting of hydrogen, a glucose, a rhamnose, a galactose, a xylose, an arabinose, a tetra-saccharide, a penta-saccharide, and a hexa-saccharide, wherein the tetra-saccharide, penta-saccharide, and hexa-saccharide are each composed of monosaccharides selected from glucose, rhamnose, galactose, xylose and arabinose (wherein the stereo configuration at C-25 is either R (rectus) form or S (sinister) form); R₂ is hydrogen and methyl group, and R₃ is one selected from the group consisting of hydrogen, a glucose, a rhamnose, a galactose, a xylose, and an arabinose.

According to the invention, the furost-5-ene-3,22,26-triol glycoside of Formula I is (25R)-26-O-beta-D-glucopyranosyl-22-hydroxy-5-ene-furostan-3beta, 26-diol-3-O-alpha-L-rhamnopyranosyl-(1→4)-alpha-L-rhamnopyranosyl-(1→4)-[alpha-L-rhamnopyranosyl-(1→2)]-beta-D-glucopyranoside; 26-O-beta-D-glucopyranosyl-22alpha-methoxy-(25S)-furost-5-ene-3beta, 26-diol-3-O-alpha-L-rhamnopyranosyl-(1→4)-beta-D-glucopyranoside; 26-O-beta-D-glucopyranosyl-22alpha-methoxy-(25R)-furost-5-ene-3beta, 26-diol 3-O-alpha-L-rhamnopyranosyl-(1→4)-beta-D-glucopyranoside; or 26-O-beta-D-glucopyranosyl-22alpha-methoxy-(25R)-furost-5-ene-3beta, 26-diol 3-O-alpha-L-rhamnopyranosyl-(1→2)-alpha-L-rhamnopyranosyl-(1→4)-beta-D-glucopyranoside. In one example of the invention, the furost-5-ene-3,22,26-triol glycoside of Formula I is (25R)-26-O-beta-D-glucopyranosyl-22-hydroxy-5-ene-furostan-3beta, 26-diol-3-O-alpha-L-rhamnopyranosyl-(1→4)-alpha-L-rhamnopyranosyl-(1→4)-[alpha-L-rhamnopyranosyl-(1→2)]-beta-D-glucopyranoside, known as dichotomin ((called as “HK-06-D01”) as illustrated in Example 1 below, having the structure of formula II:

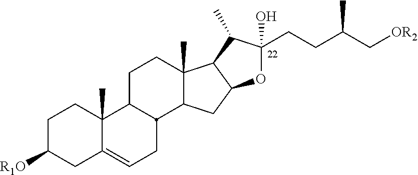

wherein R₁ is

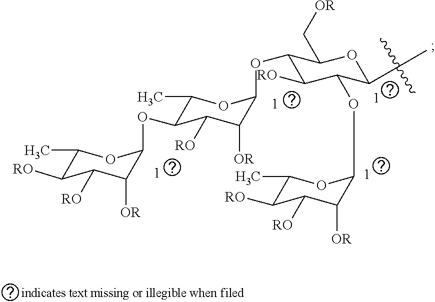

### and R₂ is

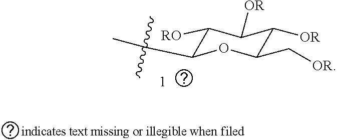

According to the invention, the furost-5-ene-3,22,26-triol glycoside of formula I may be extracted from a water extract or a partially purified composition from a plant of the Livistona or Asparagus genus as described in US Patent Publication Application, 20100179098 filed Mar. 6, 2009, published on Jul. 15, 2010, which is incorporated herein by reference in its entirety. According to the invention, the furost-5-ene-3,22,26-triol glycoside of formula I is non-toxic and relatively safe.

The term “histone deacetylase inhibitor” as used herein refers to one of the class of compounds that interfere with the function of histone deacetylase. A histone deacetylase inhibitor is proved to be one cytostatic agent that inhibit the proliferation of tumor cells in culture and in vivo by inducing cell cycle arrest, differentiation and/or apoptosis.

In one example of the invention, the histone deacetylase inhibitor is sodium butyrate (NaB), which is a short chain fatty acid that has effects at the molecular, cellular, and tissue level. It has long been known as one of histone deacetylase inhibitors. Sodium butyrate also induces growth arrest, differentiation and apoptosis in cancer cells, primarily through its effects on HDACi activity.

In another example of the invention, the histone deacetylase inhibitor is suberoylanilide hydroxamic acid (SAHA), also known as vorinostat, marketed under the name “Zolinza” for the treatment of cutaneous T cell lymphoma (CTCL), which is manufactured by Patheon, Inc. (in Canada) for Merck & Co., Inc. (in USA). Suberoylanilide hydroxamic acid (SAHA) is a member of inhibit histone deacetylases with a broad spectrum of epigenetic activities, having the systematic name: N-hydroxy-N′-phenyl-octanediamide.

The term “cancer” as used herein refers to a class of diseases in which a group of cells display uncontrolled growth, invasion (intrusion on and destruction of adjacent tissues), and sometimes metastasis (spread to other locations in the body via lymph or blood). According to the invention, the cancers to be treated include but not limited to prostate cancer, liver cancer, lung cancer and colon cancer. In one example of the invention, the cancer is prostate cancer.

The pharmaceutical composition comprises a therapeutically effective amount of the synergistic combination, depending on the mode of administration and the condition to be treated, including age, body weight, symptom, therapeutic effect, administration route and treatment time.

The pharmaceutical composition of the invention may be administered in any route that is appropriate, including but not limited to parenteral or oral administration. The pharmaceutical compositions for parenteral administration include solutions, suspensions, emulsions, and solid injectable compositions that are dissolved or suspended in a solvent immediately before use. The injections may be prepared by dissolving, suspending or emulsifying one or more of the active ingredients in a diluent. Examples of said diluents are distilled water for injection, physiological saline, vegetable oil, alcohol, and a combination thereof. Further, the injections may contain stabilizers, solubilizers, suspending agents, emulsifiers, soothing agents, buffers, preservatives, etc. The injections, are sterilized in the final formulation step or prepared by sterile procedure. The pharmaceutical composition of the invention may also be formulated into a sterile solid preparation, for example, by freeze-drying, and may be used after sterilized or dissolved in sterile injectable water or other sterile diluent(s) immediately before use.

According to the invention, the pharmaceutical composition may also be administered through oral route, wherein the composition may be in a solid or liquid form. The solid compositions include tablets, pills, capsules, dispersible powders, granules, and the like. The oral compositions also include gargles which are to be stuck to oral cavity and sublingual tablets. The capsules include hard capsules and soft capsules. In such solid compositions for oral use, one or more of the active compound(s) may be admixed solely or with diluents, binders, disintegrators, lubricants, stabilizers, solubilizers, and then formulated into a preparation in a conventional manner. When necessary, such preparations may be coated with a coating agent, or they may be coated with two or more coating layers. On the other hand, the liquid compositions for oral administration include pharmaceutically acceptable aqueous solutions, suspensions, emulsions, syrups, elixirs, and the like. In such compositions, one or more of the active compound(s) may be dissolved, suspended or emulsified in a commonly used diluent (such as purified water, ethanol or a mixture thereof, etc.). Besides such diluents, said compositions may also contain wetting agents, suspending agents, emulsifiers, sweetening agents, flavoring agents, perfumes, preservatives and buffers and the like.

On the other hand, the invention provides a method for treating a cancer comprising administering to a subject in need thereof a therapeutically effective amount of a synergistic combination of a histone deacetylase inhibitor and a furost-5-ene-3,22,26-triol glycoside of formula I with a pharmaceutically acceptable carrier, simultaneously or subsequently.

The present invention will now be described more specifically with reference to the following embodiments. It is to be noted that the following descriptions of preferred embodiments of this invention are presented herein for purpose of illustration and description only; it is not intended to be exhaustive or to limit the scope of the invention to the precise form disclosed.

### EXAMPLES

### Example 1

### Preparation of (25R)-26-O-beta-D-glucopyranosyl-22-hydroxy-5-ene-furostan-3beta, 26-diol-3-O-alpha-L-rhamnopyranosyl-(1→4)-alpha-L-rhamnopyranosyl-(1→4)-[alpha-L-rhamnopyranosyl-(1→2)]-beta-D-glucopyranoside (HK-06-D01)

One of the furost-5-ene-3,22,26-triol glycosides of formula I, (25R)-26-O-beta-D-glucopyranosyl-22-hydroxy-5-ene-furostan-3beta, 26-diol-3-O-alpha-L-rhamnopyranosyl-(1→4)-alpha-L-rhamnopyranosyl-(1→4)-[alpha-L-rhamnopyranosyl-(1→2)]-beta-D-glucopyranoside, known as dichotomin (called as “HK-06-D01” herein), was prepared following the preparation as described in the examples of US Patent Publication Application, 20100179098, which is incorporated herein by reference in its entirety. Briefly, two hundred grams of seeds of Livistona chinensis without shell were milled and boiled in 0.8 L of water for 2 hours to extract the active ingredients. The boiling/extraction was repeated for a total of 3 times. The extraction supernatants were centrifuged and concentrated under reduced pressure to a volume of 200 ml. The concentrated supernatant was extracted separately with ethyl acetate (200 ml, 3 times) and n-butanol (200 ml, 3 times). An n-butanol soluble product of 2.34 g (i.e. PK-1-1) and a water soluble product of 13.78 g (i.e. PK-1-2) were obtained after evaporation under vacuum. A portion of the n-butanol soluble product (i.e. PK-1-1, 1.55 g) was separated with centrifugal partition chromatography (CPC) using a solvent system of n-butanol-methanol-water (4:1:4), first with the organic phase as the mobile phase, then with the water phase as the mobile phase. Subsequent separation of a fraction from n-butanol elution on a Sephadex LH-20 column (140 ml, MeOH:H₂O=1:1) gave the fraction containing HK-06-D01, which was further fractionated by chromatography and purified to obtain the compound HK-06-D01. The compound was dissolved in DMSO at a stock solution of 10 mM, and fresh stock solution in DMSO was made for each experiment.

### Example 2

### Anticancer Assay for the Combination of NaB or SAHA, and HK-06-D01

1. Materials and Preparation

Sodium butyrate, also presented as NaB (Sigma Chemical Company, USA) was dissolved in water at a stock concentration of 100 mM. Fresh dilutions were made from the stock solution for each experiment. Suberoylanilide Hydroamic Acid, also presented as SAHA and known as Vorinostat (Cayman Chemical, USA) was dissolved in DMSO at a stock concentration of 75.3 mM. Fresh dilutions were made from the stock solution for each experiment.

Human prostatic carcinoma PC-3 cells (ATCC, CRL-1435, USA) were used for apoptosis assay.

The Incubation buffer includes F12K, 10% FBS at pH 7.4.

The Agonist stock was prepared by adding 5 μg Fas ligand (CD95L) to 50 μl sterile H₂O, and further adding 400 μl buffer, which stock concentration was 11.1 μg/ml (278 nM).

The Antagonist stock was prepared by adding 5 μg Fas ligand (CD95L) inhibitor in 50 μl sterile H₂O, and further adding 62.6 μl buffer, which stock concentration was 444 μg/ml (7.4 μM).

2. Apoptosis Assay

The apoptosis test was conducted following the method as described by Guseva et al. (Guseva et al., Contribution of death receptor and mitochondrial pathways to Fas-mediated apoptosis in the prostatic carcinoma cell line PC3, The Prostate. 51(4): 231-240, 2002).

The cell death of each test was detected by ELISA following the method as provided by Holdenrieder et al. (Holdenrieder et al., Quantification of nucleosomes in serum by the cell death detection ELISA^{plus}. Roche Applied Science Biochemica. 1: 25-27, 2002). The cells were collected after incubation, and then lysed and evaluated for nucleosome levels by ELISA kit (Cell Death Detection ELISA^{PLUS} Kit, Roche, 1774425). The read absorbance at 405 nm was measured for each test compound or a vehicle and the values of apoptosis % were calculated.

In Antagonist mode, NaB at a concentration of 0.3 mM, 0.6 mM, 1 mM or 3 mM or a vehicle (0.4% DMSO) as control was added to each well of the plate, and the cells (1×10⁶/ml) were also added into each well, and then preincubated for 20 min. at 37° C., 5% CO₂. Assay Reference FasL (3 nM) was added to the control wells (named as Total wells) and the Incubation buffer to the Blank wells. HK-06-D01 at a concentration of 8 μM or 3 μM was added to each well, and the plate was then incubated in Antagonist buffer for 16 hours at 37° C., 5% CO₂. The nucleosome level of each well was determined by the cell death detection kit.

In Agonist mode (wherein only one test compound was used), HK-06-D01 at a concentration of 8 μM or 3 μM, or a vehicle as control was added to each well, Assay Reference FasL (3 nM) was added to each well and Control well (named as Total wells) and the Incubation buffer to the Blank wells. The plate was then incubated in Agonist buffer for 16 hours at 37° C., 5% CO₂. The nucleosome level of each well was determined by the cell death detection kit.

In the assays for SAHA and HK-06-D01, SAHA at a concentration of 0.1 μM, 1 μM, 10 μM, 100 μM or 300 μM was replaced for the NaB. The HK-06-D01 at a concentration of 4.5 μM was used.

3. Data Analysis:

Results are expressed as a percentage of the group treated with the test compound as compared to that of the control group. The level of apoptosis (Apoptosis %) for each test compound at each concentration was calculated with the formula below:

Apoptosis %=[(c−a)/(b−a)]×100

a: average measurement of absorbance at 405 nm of nonspecific signal (vehicle control; DMSO);
b: average measurement of absorbance at 405 nm of total signal (3 nM reference compound, Fas Ligand, Oncogene, USA);
c: measurement of absorbance in the presence of a test or a reference compound.

The EC₅₀ value was determined with non-linear regression analysis of each experiment using Prism software (Graphpad Software Inc., San Deigo, Calif.). The level of Apoptosis 100% were defined as the total signal value of the 3 nM Fas Ligand-induced DNA fragmentation, and the level of Apoptosis 0% was defined as the signal value of a blank, vehicle control.

4. Results

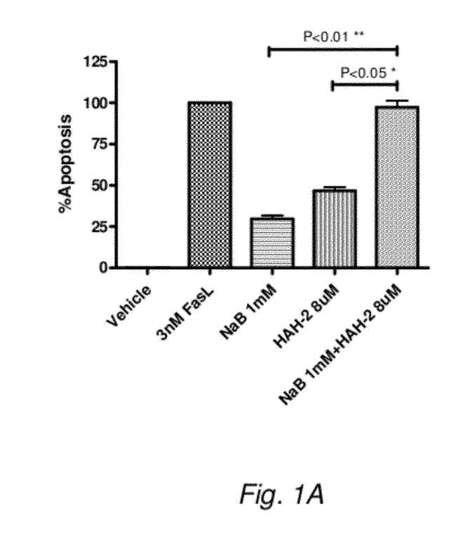

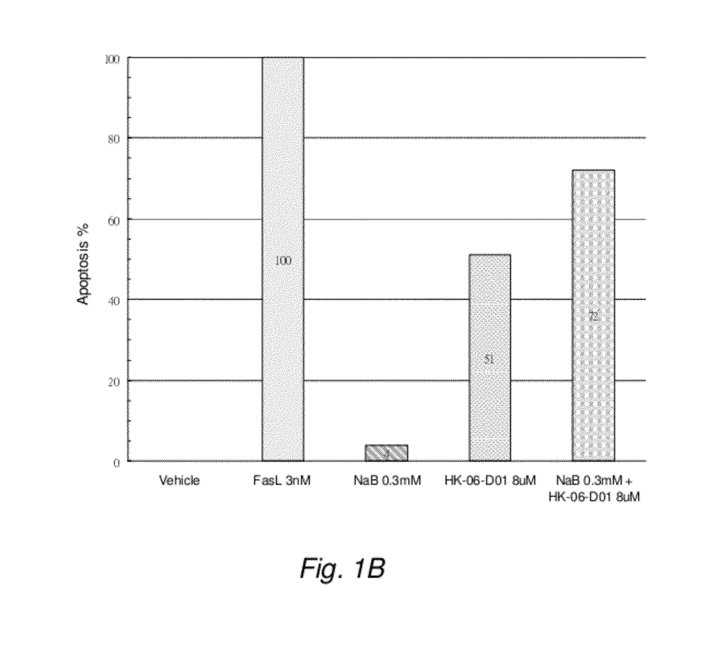

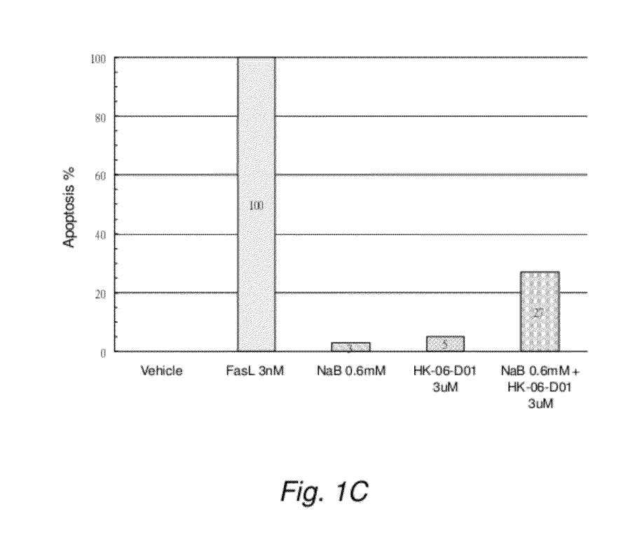

When the cells were treated with a combination of 1 mM NaB and 8 uM HK-06-D01, the means of Apoptosis % was 97.33%, significantly more than the means of Apoptosis % being 29.67% when the cells were treated with 1 mM NaB only (p<0.01), or that of Apoptosis % being 46.67% when the cells were treated with 8 uM HK-06-D01 only (p<0.05) (see FIG. 1A). The means of Apoptosis % was 72% when the cells were treated with a combination of 0.3 mM NaB and 8 uM HK-06-D01, as compared with the means of Apoptosis % being 4% when the cells were treated with 0.3 mM NaB only, or that of Apoptosis % being 51% when the cells were treated with 8 uM HK-06-D01 only (see FIG. 1B). The efficacy of the combination of NaB and HK-06-D01 was much high than either of that of NaB by 18 times, or that of HK-06-D01 by 1.4 times. The means of Apoptosis % was 27% when the cells were treated with a combination of 0.6 mM NaB and 3 uM HK-06-D01, as compared with the means of Apoptosis % being 3% when the cells were treated with 0.3 mM NaB only, or that of Apoptosis % being 5% when the cells were treated with 8 uM HK-06-D01 only (see FIG. 1C).

As shown in FIGS. 1A, 1B and 1C, it was indicated that the combination of NaB and HK-06-D01 provided a synergistic efficacy in inducing apoptosis, as compared with the efficacy of either NaB alone or HK-06-D01 alone.

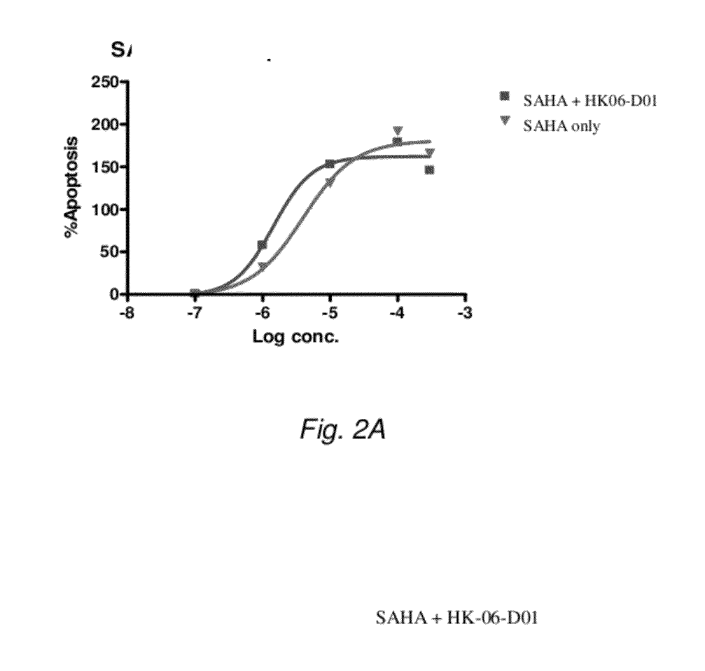

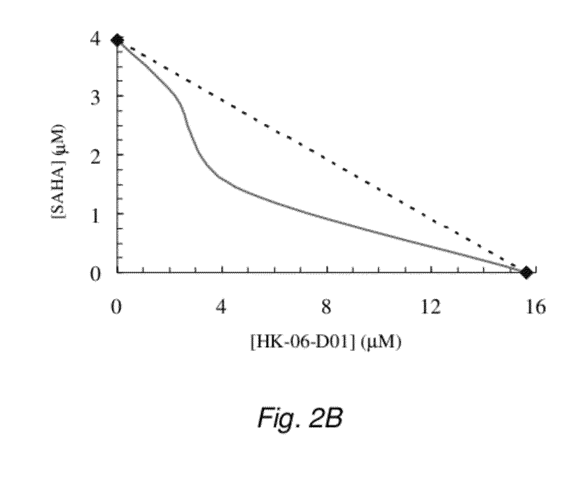

The dose response in Apoptosis % for each of SAHA and the combination of SAHA and HK-06-D01 was determined and plotted in FIG. 2A, which indicated that the EC50 value of SAHA was enhanced by adding HK-06-D01, the value of the combination of SAHA and HK-06-D01 was much more than the value of SAHA by 2.73 times.

As also shown in the isobologram in FIG. 2B, it was concluded that the combination of SAHA and HK-06-D01 provided a synergistic efficacy in inducing apoptosis in cancer cells, much more than an additive efficacy.

It is believed that a person of ordinary knowledge in the art where the present invention belongs can utilize the present invention to its broadest scope based on the descriptions herein with no need of further illustration. Therefore, the descriptions and claims as provided should be understood as of demonstrative purpose instead of limitative in any way to the scope of the present invention.

I/we claim:

1. A pharmaceutical composition for the treating a cancer comprising a synergistic combination of a histone deacetylase inhibitor and a furost-5-ene-3,22,26-triol glycoside having a structure shown in formula I: 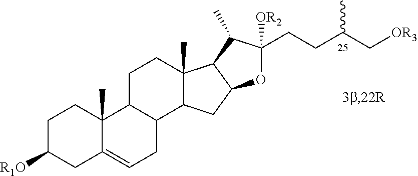
wherein R₁ is one selected from the group consisting of hydrogen, a glucose, a rhamnose, a galactose, a xylose, an arabinose, a tetra-saccharide, a penta-saccharide, and a hexa-saccharide, wherein the tetra-saccharide, penta-saccharide, and hexa-saccharide are each composed of monosaccharides selected from glucose, rhamnose, galactose, xylose and arabinose (wherein the stereo configuration at C-25 is either R (rectus) form or S (sinister) form); R₂ is hydrogen and methyl group, and R₃ is one selected from the group consisting of hydrogen, a glucose, a rhamnose, a galactose, a xylose, and an arabinose.

2. The pharmaceutical composition of claim 1, wherein the histone deacetylase inhibitor is sodium butyrate (NaB) or suberoylanilide hydroxamic acid (SAHA).

3. The pharmaceutical composition of claim 1, wherein the furost-5-ene-3,22,26-triol glycoside of Formula I is (25R)-26-O-beta-D-glucopyranosyl-22-hydroxy-5-ene-furostan-3beta, alpha-L-rhamnopyranosyl-(1→4)-alpha-L-rhamnopyranosyl-(1→4)-[alpha-L-rhamnopyranosyl-(1→2)]-beta-D-glucopyranoside; 26-O-beta-D-glucopyranosyl-22alpha-methoxy-(25S)-furost-5-ene-3beta, 26-diol-3-O-alpha-L-rhamnopyranosyl-(1→4)-beta-D-glucopyranoside; 26-O-beta-D-glucopyranosyl-22alpha-methoxy-(25R)-furost-5-ene-3beta, 26-diol 3-O-alpha-L-rhamnopyranosyl-(1→4)-beta-D-glucopyranoside; or 26-O-beta-D-glucopyranosyl-22alpha-methoxy-(25R)-furost-5-ene-3beta, 26-diol 3-O-alpha-L-rhamnopyranosyl-(1→2)-alpha-L-rhamnopyranosyl-(1→4)-beta-D-glucopyranoside.

4. The pharmaceutical composition of claim 1, wherein the furost-5-ene-3,22,26-triol glycoside of Formula I is (25R)-26-O-beta-D-glucopyranosyl-22-hydroxy-5-ene-furostan-3beta, 26-diol-3-O-alpha-L-rhamnopyranosyl-(1→4)-alpha-L-rhamnopyranosyl-(1→4)-[alpha-L-rhamnopyranosyl-(1→2)]-beta-D-glucopyranoside.

5. The pharmaceutical composition of claim 1, wherein the cancer is prostate cancer, liver cancer, lung cancer or colon cancer.

6. The pharmaceutical composition of claim 1, wherein the cancer is prostate cancer.

7. The pharmaceutical composition of claim 1, wherein the histone deacetylase inhibitor is sodium butyrate (NaB) or suberoylanilide hydroxamic acid (SAHA), and the furost-5-ene-3,22,26-triol glycoside of Formula I is (25R)-26-O-beta-D-glucopyranosyl-22-hydroxy-5-ene-furostan-3beta, 26-diol-3-O-alpha-L-rhamnopyranosyl-(1→4)-alpha-L-rhamnopyranosyl-(1→4)-[alpha-L-rhamnopyranosyl-(12)]-beta-D-glucopyranoside.

8. The pharmaceutical composition of claim 1, wherein the histone deacetylase inhibitor is sodium butyrate (NaB), and the furost-5-ene-3,22,26-triol glycoside of Formula I is (25R)-26-O-beta-D-glucopyranosyl-22-hydroxy-5-ene-furostan-3beta, 26-diol-3-O-alpha-L-rhamnopyranosyl-(1→4)-alpha-L-rhamnopyranosyl-(1→4)-[alpha-L-rhamnopyranosyl-(1→2)]-beta-D-glucopyranoside.

9. The pharmaceutical composition of claim 1, wherein the histone deacetylase inhibitor is suberoylanilide hydroxamic acid (SAHA), and the furost-5-ene-3,22,26-triol glycoside of Formula I is (25R)-26-O-beta-D-glucopyranosyl-22-hydroxy-5-ene-furostan-3beta, 26-diol-3-O-alpha-L-rhamnopyranosyl-(1→4)-alpha-L-rhamnopyranosyl-(1→4)-[alpha-L-rhamnopyranosyl-(1→2)]-beta-D-glucopyranoside.

10. A method for treating a cancer comprising administering to a subject in need thereof a therapeutically effective amount of a synergistic combination of a histone deacetylase inhibitor and a furost-5-ene-3,22,26-triol glycoside of formula I as defined in claim 1 with a pharmaceutically acceptable carrier, simultaneously or subsequently.

11. The method of claim 10, wherein the histone deacetylase inhibitor is sodium butyrate (NaB) or suberoylanilide hydroxamic acid (SAHA).

12. The method of claim 10, wherein the furost-5-ene-3,22,26-triol glycoside of Formula I is (25R)-26-O-beta-D-glucopyranosyl-22-hydroxy-5-ene-furostan-3beta, 26-diol-3-O-alpha-L-rhamnopyranosyl-(14)-alpha-L-rhamnopyranosyl-(1→4)-[alpha-L-rhamnopyranosyl-(1→2)]-beta-D-glucopyranoside; 26-O-beta-D-glucopyranosyl-22alpha-methoxy-(25S)-furost-5-ene-3beta, 26-diol-3-O-alpha-L-rhamnopyranosyl-(1→4)-beta-D-glucopyranoside; 26-O-beta-D-glucopyranosyl-22alpha-methoxy-(25R)-furost-5-ene-3beta, 26-diol 3-O-alpha-L-rhamnopyranosyl-(1→4)-beta-D-glucopyranoside; or 26-O-beta-D-glucopyranosyl-22alpha-methoxy-(25R)-furost-5-ene-3beta, 26-diol 3-O-alpha-L-rhamnopyranosyl-(1→2)-alpha-L-rhamnopyranosyl-(1→4)-beta-D-glucopyranoside.

13. The method of claim 10, wherein the furost-5-ene-3,22,26-triol glycoside of Formula I is (25R)-26-O-beta-D-glucopyranosyl-22-hydroxy-5-ene-furostan-3beta, 26-diol-3-O-alpha-L-rhamnopyranosyl-(1→4)-alpha-L-rhamnopyranosyl-(1→4)-[alpha-L-rhamnopyranosyl-(1→2)]-beta-D-glucopyranoside.

14. The method of claim 10, wherein the cancer is prostate cancer, liver cancer, lung cancer and colon cancer.

15. The method of claim 10, wherein the cancer is prostate cancer.

16. A method for the treating a cancer comprising administering to a subject in need thereof a therapeutically effective amount of a synergistic combination of a histone deacetylase inhibitor and (25R)-26-O-beta-D-glucopyranosyl-22-hydroxy-5-ene-furostan-3beta, 26-diol-3-O-alpha-L-rhamnopyranosyl-(1→4)-alpha-L-rhamnopyranosyl-(1→4)-[alpha-L-rhamnopyranosyl-(1→2)]-beta-D-glucopyranoside.

17. The method of claim 16, wherein the histone deacetylase inhibitor is sodium butyrate (NaB).

18. The method of claim 16, wherein the histone deacetylase inhibitor is suberoylanilide hydroxamic acid (SAHA).

19. The method of claim 16, wherein the cancer is prostate cancer, liver cancer, lung cancer or colon cancer.

20. The method of claim 16, wherein the cancer is prostate cancer.
